(19) United States
(12) Patent Application Publication
(10) Pub. No.: US 2022/0325212 A1
(43) Pub. Date: Oct. 13, 2022

(54) YEAST CULTURE MEDIUM AND METHOD FOR PRODUCING ALCOHOLIC BEVERAGES

(71) Applicant: Sundaze I, Inc., Dover, DE (US)

(72) Inventor: Juan Diego Cordón Toledano, Emeryville, CA (US)

(21) Appl. No.: 17/709,351

(22) Filed: Mar. 30, 2022

Related U.S. Application Data
(60) Provisional application No. 63/167,805, filed on Mar. 30, 2021.

(51) Int. Cl. C12G 3/024; C12N 1/16; C12G 3/04; C12H 6/00

(52) U.S. Cl. CPC C12G 3/024; C12N 1/16; C12G 3/04; C12H 6/00

(57) ABSTRACT

The present application describes improved yeast culture media that are particularly suitable for cultivation of Pichia yeast, in particular P. kluyveri. Also described herein are methods of cultivating yeast using this improved culture medium. Further described herein are improved methods of producing a fermented juice beverage.

### CROSS-REFERENCE TO RELATED APPLICATIONS

This application claims the benefit of U.S. Provisional Application 63/167,805 filed Mar. 30, 2021, the entire contents of which are hereby incorporated by reference in its entirety.

### FIELD

Described herein are improved yeast culture media which optimize the growth and activity of yeast, in particular Pichia kluyveri, and method employing such culture media and yeast such as P. kluyveri to produce a juice-based alcoholic beverage as well as similar non-alcoholic beverages and dietary supplements.

### BACKGROUND

Species of the genus Pichia, such as P. kluyveri and P. fermentans, are responsible for the deterioration of food, including fruits such as pears, tomatoes, oranges, etc. European patent publication EP2641962 B1 describes a method for preparing a fermented orange juice beverage using P. kluyveri, in particular the strain of P. kluyveri 4N187 deposited with the Spanish Type Culture Collection (CECT) on 12 May 2010 with accession number CECT 13055. The entire contents of EP2641962 are incorporated herein by reference. P. kluyveri is able to ferment simple sugars such as glucose or fructose but not complex sugars, such as galactose, sucrose, maltose, raffinose or trehalose. This permits the use of the organism for fermentation of food without the loss of many of the sugars present in the food, which are therefore able to naturally sweeten the resulting product. Other useful technology is found in KR101288799 (methods of producing fermented citrus product and increasing flavinoid content), CN102994919 (fermented citrus wine and production technology) and CN109770139 (methods of production of low alcohol fermented beverages), which are hereby incorporated by reference in their entirety.

The sugar profile in Orange Juice is typically comprised of 50% Sucrose, 25% Glucose and 25% Fructose on average. The preferred strain of P. kluyveri is Invertase (−) so it is unable to metabolize the sucrose. In an ideal fermentation, all the sucrose will be leftover in the fermented orange juice and will in part account for the sweetness in the finished product.

In addition, this strain of P. kluyveri is Crabtree-Negative. Oxygen is required to ferment. When no aeration/agitation is provided (no DO in the wort), the yeast will yield a max of 1.5% ABV over 10-15 days. It will create a film over the juice and will produce a high concentration of esters in the juice. On the other hand, if the wort's DO is in saturation levels, the yeast will promote growth and will minimize the production of alcohol. In a propagation scenario with aeration and agitation at about 30° C., the yeast is often able to consume all the fermentable sugars within 48 hours. Common fermentation characteristics:
a. Enhance fruit flavors (thiols, terpenes, esters)
b. Very low volatile phenols
c. Very low H2S
d. Low production of acetic acid
e. Mouth feel
f. Medium production of polysaccharides
g. Low production of SO2

Fruit juice, such as orange juice can be received for processing in several ways, including but not limited to; aseptic tote(s), flexitank or tanker. When receiving totes, it can be placed into refrigerated storage (34-38 F) as soon as possible. If receiving in a large volume format and production is not slated within 36 hours of receival, the receiving tank is refrigerated to persevere freshness and decrease spoilage. The juice can also be inoculated with a yeast such as Pichia kluyveri prior to or during shipment to limit the growth of unhelpful microorganisms.

Juices, such as orange juice are full of native yeast and bacteria, so rather than employ post-fermentation sterilizing practices, the focus can be simply on microbial management to avoid having common yeast types out-complete the Pichia strain used for fermentation.

### SUMMARY

The present application describes improved yeast culture media that are particularly suitable for cultivation of Pichia yeast, in particular P. kluyveri. Also described herein are methods of cultivating yeast using this improved culture medium. Further described herein are improved methods of producing a fermented juice beverage.

### BRIEF DESCRIPTION OF THE DRAWINGS

FIG. 1 shows the process flow diagram for Tunnel Pasteurization.

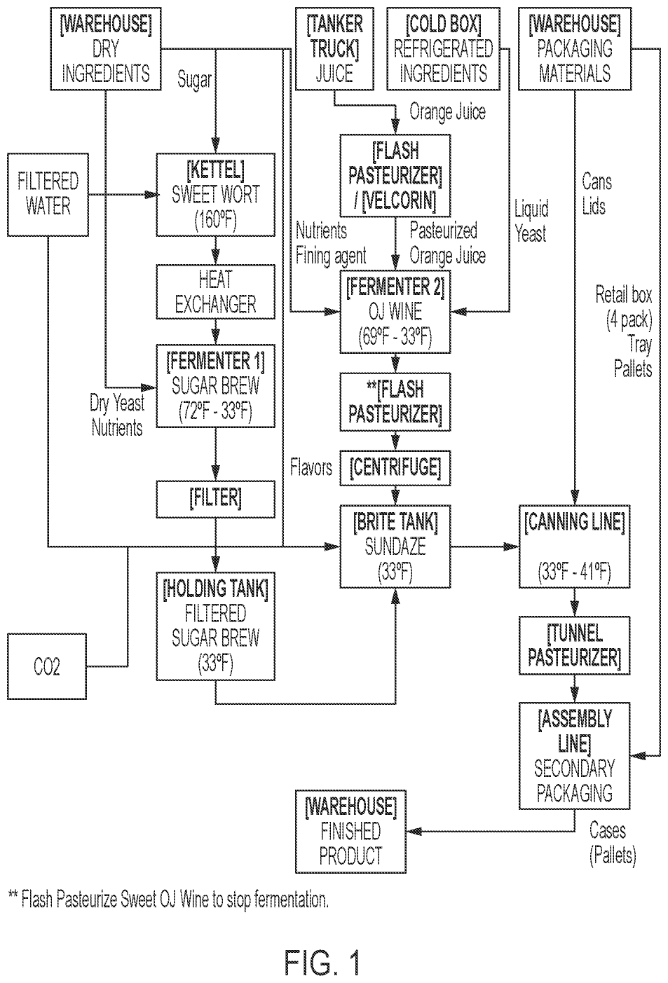

FIG. 2 shows the process flow diagram for Flash Pasteurization and Velcorin.

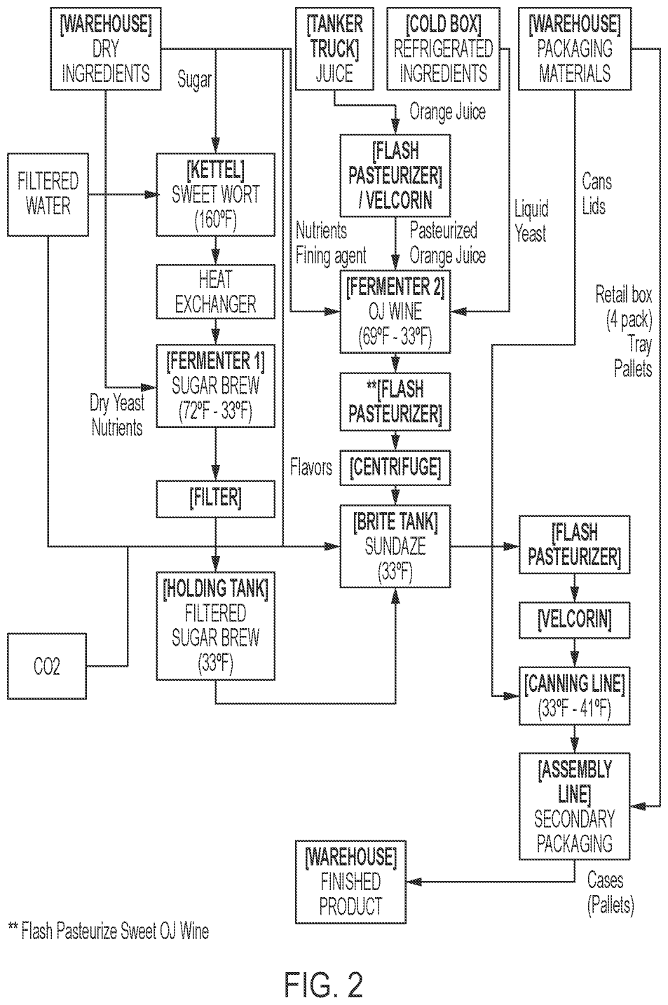

FIG. 3 shows an exemplary ideal fermentation curve showing sugar levels, pH, ABV and temperatures over the course of fermentation.

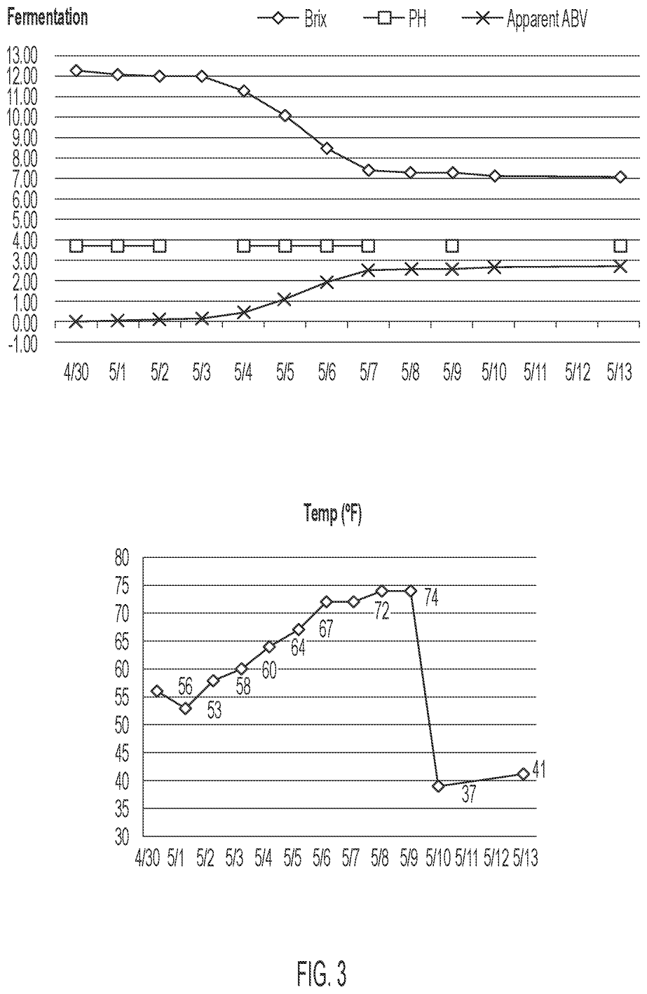

### DETAILED DESCRIPTION

### Definitions

“ABV” means Alcohol By Volume, a measure of alcohol concentration.

“Brix” is a measure of the sugar content of an aqueous solution, commonly used to specify the sugar content of wine. One degree Brix is equivalent to a concentration of 1 gram of sucrose in 100 grams of solution.

“Citrus juice” is the juice of a citrus fruit. As used herein, the term encompasses juice of a single type of citrus fruit, and/or a blend of juices from different citrus fruits. Exemplary citrus fruits include, but are not limited to, amanatsu, balady citron, bergamot orange, bitter orange, Seville orange, blood orange, buddhas hand, bushukan, calmondin, cam sánh, chinotto, citrange, citron, citrumelo, clementine, Corsican citron, desert lime, etrog, finger lime, Florentine citron, grapefruit, Greek citron, haruka, hassaku, hyganatsu, konatsu, tosakonatsu, New Summer orange, adadomiakan, jabara, kabosu, kaffir lime, kanpei, kawachi bankan, key lime, kinkoji unshiu, kinnow, kyomi, kobayashi mikan, Koji orang, Kuchinotsu No. 37, kumquat, lemon, lime, mandarin orange, mangashanyegan, meyer lemon, Moroccan citron, myrtle-leaved orange, orange, oroblanco, papeda, persian lime, pomelo, pomia, ponkan, ponderosa lemon, rangpur, round lime, Australian lime, satsuma, shangjuan, ichan lemon, Shonan Gold, sudachi, sweet limetta, Taiwan tangerine, tangelo, tangerine, tangor, ugli fruit, Volkamer lemon, yukou, and yuzu.

“Juice” may be pure juice from a single type of fruit, or a mixture of juices, it can be fresh juice or reconstituted form concentrate. In one alternative embodiment, the juice is reconstituted from a juice concentrate by adding water prior to fermentation. In an alternative embodiment, the juice is at its natural concentration (i.e., not from concentrate). As used herein, “juice” also encompasses a mixture of juice and fruit solids (also referred to as “must”).

Pichia kluyveri 4N187 is a strain of yeast deposited in the Spanish Type Culture Collection (CECT) with accession number 13055.

“Plato” is a measure of sugar content of an aqueous solution, commonly used to specify the residual sugar content in beer. One degree Plato is equivalent to a concentration of 1 gram of sucrose in 100 grams of solution.

### Exemplary Growth Medium

Provided herein is a growth medium especially useful for propagating Pichia yeast, such as Pichia kluyveri. The medium comprises equal parts citrus juice and water; 34 g/l of glucose; and 34 g/l of fructose; in one embodiment, the citrus juice is orange juice. The pH of the growth medium is between 3 and 5, for example between 3.3 and 4.6. The concentration of sugars in the growth medium is about 11 brix or 11 plato. The growth medium may be supplemented with up to about 0.5 g/l of a complex yeast nutrient mixture. Such a yeast nutrient mixture may comprise one or more of the following ingredients: one or more assimilable amino acids from inactivated and autolyzed yeast; one or more sterols; one or more unsaturated fatty acids; magnesium sulfate; thiamine; folic acid; niacin; calcium pantothenate; diammonium phosphate; and Inactivated yeast.

### Exemplary Process for Culturing Pichia Yeast

Also described herein is a method of culturing Pichia yeast, such as Pichia kluyveri, comprising adding the yeast to a culture medium to form a culture of the yeast. One suitable strain of P. kluyveri is P. kluyveri 4N187. One culture medium comprises equal parts citrus juice and water; 34 g/l of glucose; and 34 g/l of fructose; in one embodiment, the citrus juice is orange juice, other flavoring and beneficial agents such as ginger, rosemary, lavender, Jalapeno/Thai Chili flakes, sweet orange wine, pomegranate etc. can also be added before, during, or after fermentation. The citrus juice can be pasteurized before adding to the media. The pH of the growth medium is between 3 and 5, for example between 3.3 and 4.6. The concentration of sugars in the growth medium is about 11 brix or 11 plato. The growth medium may be supplemented with up to about 0.5 g/l of a complex yeast nutrient mixture. Such a yeast nutrient mixture may comprise one or more of the following ingredients: one or more assimilable amino acids from inactivated and autolyzed yeast; one or more sterols; one or more unsaturated fatty acids; magnesium sulfate; thiamine; folic acid; niacin; calcium pantothenate; diammonium phosphate; and Inactivated yeast. A commercial mix sold as Fermaid K is also suitable. In the method described herein, the culture medium may be maintained at about 30° C. In one alternative embodiment, the culture medium is aerated with sterile filtered air. In an alternative embodiment, the culture medium is physically agitated. In another alternative embodiment, the culture medium is both aerated and physically agitated. In another alternative embodiment, the mixture is neither aerated nor agitated. Physical agitation of the culture medium may be accomplished using any suitable means; in one exemplary embodiment, the culture medium is agitated by stirring at 100-300 rpm. The culturing process may be terminated when the fermentable sugar content of the culture is sufficiently depleted. In one exemplary embodiment, the culturing process is terminated when the sugar content of the culture is about 4 plato. In an alternative embodiment, the culturing process may be terminated when the pH of the culture is about 3.3. Alternatively, propagation may be terminated when the yeast concentration in the medium reaches about 0.5 billion to about 1 billion yeast cells per ml; for example, about 0.8 billion cells per ml.

The culturing process described herein may comprise a single or multiple cycles of propagation. In such an embodiment, following the first cycle of propagation, the culture is refreshed with new culture media when the sugar content of the mixture is depleted to about 4 plato and the pH of the culture reaches about 3.3. In one embodiment, each cycle lasts between about 24 and about 48 hours; for example; about 36 hours. Alternatively, each cycle may be terminated when the yeast concentration in the medium reaches about 0.5 billion to about 1 billion yeast cells per ml; for example, about 0.8 billion cells per ml.

### Exemplary Methods of Producing Alcoholic and Non-Alcoholic Beverages

Also described herein is a method for producing an alcoholic beverage using juice with Pichia kluyveri yeast and performing alcoholic fermentation of the juice/yeast mixture under aerobic conditions. In one embodiment, the juice is orange juice.

In the method described herein, the fermentation proceeds under conditions of controlled temperature. In one embodiment, the temperature of the mixture is maintained at about 20° C. The mixture may be continually aerated with sterile filtered air. Alternatively, the mixture may be physically agitated. In another alternative embodiment, the mixture may be aerated and agitated. In another alternative embodiment, the mixture is neither aerated nor agitated. The mixture may be agitated continuously. Agitation may be performed using any suitable means; in one embodiment, the mixture is agitated by mechanical stirring of the mixture. Constant amounts of O₂ can be infused into the mixture to maintain a concentration of about 6-12 ppm, preferably 9 ppm. The speed of growth is thereby maintained, which can affect the flavor profile and yeast growth as well as how fast the sugar is used as well as what types of sugar are used.

Following fermentation, ethanol may be added to increase the alcohol content of the beverage to a desired level. For example, ethanol may be added to increase the alcohol content of the beverage to between about 3.5% and about 12%. In one alternative embodiment, ethanol is added to increase the alcohol content of the beverage to about 6%. The addition of alcohol can be manipulated to kill the remaining yeast or permit further fermentation. Any source of ethanol which is suitable for human consumption, and which has an ethanol content higher than the beverage following the fermentation process, may be employed to increase the ethanol content of the beverage. Suitable sources of ethanol include distilled alcohol from orange, grain, sugar cane, or any other source with an ABV content equal to or higher than 20% ABV; or fermented alcohol from grain, cane sugar, or other fruits, preferably with an ABV content equal or less than 20% ABV.

If desired, the beverage may be fermented under conditions such that the carbon dioxide produced as a result of the fermentation process is retained in the beverage. Additional carbonation may be added to the beverage following fermentation, using known methods for carbonating alcoholic beverages. Such methods are described in, for example, European patent application publication EP2641962B1.

Following fermentation, the sweetness of the beverage may be adjusted to a desired level of sweetness by adding a natural sweetener, such as cane sugar or corn syrup, or by adding an artificial sweetener such as aspartame, or by adding other sweetening agents such as other juices, purees, concentrates, or juice powders to the beverage. Alternatively, the sweetness of the beverage may be decreased by decreasing the concentration of residual sugars in the beverage, for example, by dilution with water, or with an ethanol-water solution. In one embodiment, the concentration of residual sugars is decreased to about −1 to 7 Plato, for example, to about 2 Plato. In some embodiments, the use of P. kluyveri permits the sweetness of unfermented sucrose or other sugar to sweeten the product.

The pH of the beverage may be adjusted by adding an acid, such as citric acid or potassium bicarbonate

If desired, the beverage may be blended with natural flavors, extracts, and/or herbs or other botanicals, juices, purees, juice powders, juice concentrates, etc. Functional ingredients, such as flavonoids, probiotics, especially microencapsulated probiotics, adaptogens, and/or antioxidants may be added as desired. Dietary ingredients, such as, but not limited to, vitamins, minerals, and amino acids may be added as desired.

In an alternative embodiment, the beverage can be mixed with kombucha (alcoholic or non-alcoholic); water, ethanol, or a mixture of water and ethanol may also be added to the beverage/kombucha mixture. In alternative embodiments the alcoholic content can be reduced through fermentations well known in the art or simply removed from the product.

### Example 1

The following table illustrates an exemplary formulation range for a fermented orange juice beverage prepared according to the methods described herein. This beverage is prepared using not-from-concentrate 100% Valencia aseptic orange juice.

TABLE 1
Exemplary Specification of fermentation mixture.

| Parameters | Range | Common |
|---|---|---|
| Brix degrees | 10-15 | 11 |
| % Acidity (w/w) | 0.7-1 | 0.9 |
| Ratio (Brix/Acidity) | 12-24 | 12.5 |
| pH | 3.3-4.2 | 3.5 |
| Pulp % (vol/vol) | 10-20 | 10 |
| % Oil (vol/w) | 0.015-0.035 | 0.0189 |
| Total Aerobic Microorganisms (cfu/ml) | 0-10 | <10 |
| Yeast and Mold count (cfu/ml) | 0-10 | <10 |
| Total Coliform (cfu/ml) | 0-10 | <10 |
| Color OJ Index (USDA) | 36-38 | 37 |
| Flavor (USDA) | 36-38 | 37 |

The yeast used in this example is P. kluyveri, inoculated at a range of between 6 and 12 million cells per ml, most commonly at 8 million cells per ml. Such an inoculation of the medium or wort is sometimes referred to as pitching. Fermentation takes place at a temperature between 15° and 25° C., ideally at 20° C., under constant aeration with sterile filtered air until the target sugar depletion is reached.

The fermentation is aided by addition of a commercial yeast nutrient supplement mixture (Fermaid K™) that contains a blend of assimilable amino acids from inactivated and autolyzed yeast, sterols and unsaturated fatty acids, magnesium sulfate, thiamine, folic acid, niacin, calcium pantothenate, diammonium phosphate (DAP), and inactivated yeast. The nutrient supplement may be added at the beginning of the fermentation (day 0) or split equally, with ½ dose added at the beginning of the fermentation and the second ½ dose added upon ¼ depletion of the sugars.

Depending on the scale (volume) of the fermentation, the total juice volume to be fermented may be added at the beginning of the fermentation, or in more than one step as the desired yeast population increases and sugar is depleted. For example, for a 3,000 L fermentation, fermentation starts with 1,500 L of orange juice and the total yeast culture to be pitched together with the needed nutrients. As sugars are depleted, ideally at about ¼ of the total sugar depletion, another 1,500 L of orange juice will be added together with the needed nutrients, increasing the total fermenting volume to about 3,000 L.

Fermentation is terminated by “cold crashing”: dropping the temperature of the fermentation tank to 0-5° C. At this point, 0.05% of a fining agent (Gelocolle, Scott Laboratories) can be added to the fermented orange juice to allow solids in suspension and yeast to sediment and clarify the liquid. The liquid is allowed to rest for 1-5 days, most commonly 3 days. Other options for clarifying the liquid include one or more of centrifugation, filtration, and decantation. Following clarification, the sediments are discarded, and the clarified liquid is moved forward into the blending process.

Finally, the beverage product may be pasteurized if desired. Methods for pasteurization are well known in the art.

### Example 1

Pitch Rate: 8 Million cells/ml
Temperature: 15-20° C.
Initial brix: 11-12
Final brix: 6-7
ABV: 2.5-3%
Initial pH: 3.54
Final pH: 3.5
Time: 5-7 days

Using a fermentation media of S/S Valencia Orange Juice+Nutrients (0.0017 lb/gal of Fermaid K/Lallemand). One addition at yeast pitching. The fermentation took place with constant aeration using Sterile Filtered compressed air (125 cpm) at 5 psi, 700 gal of juice in 1,500 gal tank—no carb stone) or Sterile Filtered compressed air (0.31 cpm) at 10 psi, 350 gal of juice in 700 gal tank—no carb stone). Fermentation was cold crashed to 3° C. or less at 7 plato, aeration system was removed and Gelocolle (fining agent) was added at 0.05% v/v to help solids and yeast to sediment and clarified the juice. CO2 is used to add the Gelocolle and displace Oxygen in the juice. After at least 48 hours in conditioning, the fermented juice was racked off leaving the solids behind and it is then blended with the rest of the ingredients.

Ideal Fermentation Curves such as Table II were possible.

| | | | | Apparent | | | | | Event/ |
|---|---|---|---|---|---|---|---|---|---|
| Date | Temp F. | Brix | PH | ABV | TA | FS02 | Initials | Time | Tasting Notes |
| 4/30 | 56 | 12.30 | 3.69 | −0.05 | | | ACM | 7:40:00 | |
| | | | | | | | | AM | |
| 5/1 | 53 | 12.10 | 3.71 | 0.05 | | | ACM | 7:40:00 | |
| | | | | | | | | AM | |
| 5/2 | 58 | 12.00 | 3.72 | 0.11 | | | ACM | 7:20:00 | |
| | | | | | | | | AM | |
| 5/3 | 60 | 12.00 | | 0.11 | | | | | |
| 5/4 | 64 | 11.30 | 3.7 | 0.47 | | | | | 75 millions |
| | | | | | | | | | cells/ml |
| 5/5 | 67 | 10.10 | 3.69 | 1.10 | | | ACM | 6:50:00 | 2 billions |
| | | | | | | | | AM | cells/ml |
| 5/6 | 72 | 8.50 | 3.66 | 1.94 | | | ACM | 6:30:00 | 320 million |
| | | | | | | | | AM | cells/ml |
| 5/7 | 72 | 7.40 | 3.67 | 2.52 | | | ACM | 5:50:00 | 180 million |
| | | | | | | | | AM | cells/ml |
| 5/8 | 74 | 7.30 | | 2.57 | | | | | |
| 5/9 | 74 | 7.30 | 3.68 | 2.57 | | | ACM | 9:30:00 | crashed |
| | | | | | | | | AM | |
| 5/10 | 39 | 7.10 | | 2.68 | | | | | |
| 5/13 | 41 | 7.10 | 1.64 | 2.68 | | | ACM | 8:00:00 | |
| | | | | | | | | AM | |

In some production runs orange juice fermentation batches showed high numbers of microbiology contaminations, justifying the having the fermentations going all the way to dryness and not ending at target gravity.

Flavor/Aroma Profile for a batch is found in Table III.

| | | Deviation | | Deviation | | |
|---|---|---|---|---|---|---|
| | | from | | from | Ideal FOJ | |
| Assay | TK10 | reference | TK11 | reference | REFERENCE | Notes |
| 2,3 Pentanedione | 15 | 0.00% | 15 | 0.00% | 15 | Honey-like flavor in beer/ |
| (As-Is) (ppb) | | | | | | off-flavor |
| Diacetyl (As-Is) (ppb) | 20.04 | 33.60% | 15 | 0.00% | 15 | Artificial butter flavor/ |
| | | | | | | off-flavor |
| Diacetyl/Pentanedione | 1.336 | 33.60% | 1 | 0.00% | 1 | The higher the ratio, the |
| Ratio (ppm) | | | | | | more changes of contamination |
| 1-Propanol (ppm) | 14.54 | 94.91% | 14.5 | 94.37% | 7.46 | Fusel Alcohol, solvent-like |
| | | | | | | flavor. The lower the better |
| Acetaldehyde (ppm) | 10.47 | −61.34% | 9.84 | −63.66% | 27.08 | Tart flavor reminiscent of green |
| | | | | | | apples. Low levels is a sign of |
| | | | | | | healthier fermentation. |
| Acetone (ppm) | 1.52 | 1420.00% | 1.39 | 1290.00% | 0.1 | Fusel Alcohol, solvent-like |
| | | | | | | flavor. The lower the better |
| Ethyl Acetate (ppm) | 18.61 | −11.55% | 26.29 | 24.95% | 21.04 | Fruity aromas. More at higher |
| | | | | | | temp and lower pitch rate |
| Ethyl Butyrate (ppm) | 0.5 | 56.25% | 0.43 | 34.38% | 0.32 | Fruity, Pineapple, Tropical |
| Ethyl Hexanoate (ppm) | 0.1 | −99.82% | 0.1 | −99.82% | 54.64 | Anis |
| Ethyl Octanoate (ppm) | 0.48 | 380.00% | 0.49 | 390.00% | 0.1 | Sour Apple, Soap |
| Isoamyl Acetate (ppm) | 0.79 | −45.89% | 0.96 | −34.25% | 1.46 | Banana and Pear |
| Amyl Alcohols (ppm) | 76.92 | 115.82% | 94.08 | 163.97% | 35.64 | Fusel, spirituous |
| Isobutanol (ppm) | 17.88 | −45.15% | 20.62 | −36.75% | 32.6 | Fusel, spirituous |
| Isobutyl Acetate (ppm) | 0.1 | 0.00% | 0.1 | 0.00% | 0.1 | Fruity at low concentrations, |
| | | | | | | unpleasant at higher |
| | | | | | | concentrations |
| Methanol (ppm) | 41.67 | 15.91% | 44.56 | 23.95% | 35.95 | Produced from pectines. |
| | | | | | | White Wine 140 ppm |

### Example 2

We have investigated the effect varying the amount of Dissolved Oxygen (DO) would have on a Pichia kluyveri fermentation in orange juice. To determine the effect we tested DO at three (3) different levels, along with a control with no added oxygen and agitation, utilizing the Pichia kluyveri yeast strain and provided yeast nutrient (Lallemand Fermaid K); 4 ppm, 8 ppm, and 12 ppm. Dissolved oxygen was set by changing the gas mixture on the DASGIP DASBOX to allow for differing percentages of oxygen and then using a Hamilton ARC Bluetooth adapter to correlate those percentages to a set ppm of DO. Compressed air was provided from a sterile air tank at 6 L/h. Oxygen levels were set approximately 24 hours before inoculation to ensure stability. After inoculation, the fermenters were set to an agitation rate of 100 rpm, except the non aeration/non agitation control. Fermentations were executed in a controlled environment to avoid cross contamination in 1 L vessels with controlled temperature and controlled DO. Plato, temp, pH and DO was continuously monitored and VA, TA, Gluc/Fruc and ABV has been measured. In this set up, aeration and agitation provided constituted an ideal 1 fermentation set up for Pichia kluyveri. As observed, dryness was achieved on 30 hours average. In this example, dryness is expected to be accomplished between 5 and 7 days. Longer lag phase, temperature and less ideal aeration and agitation set up will increase fermentation time. Oxygen is vital for the growth and fermentation capacity of Pichia kluyveri. Compressed air can be pumped in at a steady amount to reach a DO of about 8 ppm. No significant differences has been observed at different DO concentrations studied. However, best organoleptic results were obtained at higher DO concentrations: for example, about 12 ppm. Agitation was needed to keep yeast in suspension and avoid forming a biofilm on top of the juice. Either heavy aeration from the bottom of the fermenter with big bubbles or pump over (2-3 times per day) in addition to the constant aeration were options at scale when agitation is not available in the fermenter. Pump-overs introduce extra oxygen which is beneficial to the growth and fermentation capacity of Pichia kluyveri. After lag phase (9 hours in this experimentation), DO readings were zero even with constant air supply and agitation. The yeast was using all available oxygen, even after fermentable sugars depletion. Alcohol concentration depletion was observed after the yeast consumed all fermentable sugars in the orange juice (glucose and fructose). After dryness (glucose and fructose full depletion), the cell count starts decreasing along with an increase on pH. Besides tracking glucose and fructose, pH can also be an indicator of dryness since it starts increasing once simple sugars have been depleting. Sucrose tracking was found to be a good indicator for cross contamination. Any depletion of sucrose indicates a spoilage microorganism was present and active in the fermentation. During an ideal fermentation, pitched at 8*10\hat{ }6 cell/ml, yeast growth and normalized between 5 and 6.5*10\hat{ }8 cells/ml, keeping high yeast viability during the fermentation (above 80%).

What is claimed is:

1. A growth medium for propagating Pichia yeast, the medium comprising:
equal parts orange juice and water;
34 g/l of glucose; and
34 g/l of fructose
wherein the pH of the growth medium is 3.3-4.6; and
wherein the concentration of sugars in the growth medium is about 11 brix or 11 plato.

2. The growth medium of claim 1, further comprising 0.5 g/l of a complex yeast nutrient comprising
one or more assimilable amino acids from inactivated and autolyzed yeast;
one or more sterols;
one or more unsaturated fatty acids;
magnesium sulfate;
thiamine;
folic acid;
niacin;
calcium pantothenate;
diammonium phosphate; and
Inactivated yeast.

3. A method of culturing Pichia yeast comprising:
Adding the yeast to a culture medium according to claim 1 to form a culture;
maintaining culture at 30° C.;
without aeration; or aerating the culture with sterile filtered air, physically agitating the culture, or both.

4. The method of claim 3, wherein the agitating is performed with a rotary mixer which mixes the culture at 100-300 rpm.

5. The method of claim 3, wherein the culturing is terminated when the sugar content of the culture is depleted to about 4 plato and/or the pH of the culture is about 3.3.

6. The method of claim 3, wherein the method comprises multiple cycles of fermentation.

7. The method of claim 6, wherein each cycle lasts between about 24 and about 48 hours

8. The method of claim 6, wherein each cycle is terminated when the sugar content of the mixture is depleted to about 4 plato and the pH of the medium reaches about 3.3.

9. The method of claim 6, wherein each cycle is terminated when the yeast concentration in the medium reaches about 0.5 billion to about 1 billion cells per ml.

10. The method of claim 9, wherein the yeast concentration reaches about 0.8 billion cells per ml.

11. The method of claim 1, wherein the yeast is Pichia kluyveri.

12. The method of claim 11, wherein the yeast is P. kluyveri 4N187.

13. A method for producing an alcoholic beverage comprising pitching citrus juice with Pichia kluyveri yeast, and performing alcoholic fermentation of the juice/yeast mixture under aerobic conditions.

14. The method of claim 13, wherein the citrus juice is orange juice.

15. The method of claim 13, wherein the fermentation proceeds under conditions of controlled temperature.

16. The method of claim 15, wherein the temperature of the mixture is maintained at about 20° C.

17. The method of claim 13, further comprising continually aerating the mixture with sterile filtered air.

18. The method of claim 13, further comprising the step of agitating the mixture.

19. The method of claim 18, wherein the mixture is agitated continuously.

20. The method of claim 13, further comprising the step of adding ethanol to increase the alcohol content of the beverage to between about 3.5% and about 12%.

21. The method of claim 20, wherein the alcohol content of the beverage is increased to about 6%.

22. The method of claim 13, further comprising the step of carbonating the mixture.

23. The method of claim 13, further comprising the step of decreasing the concentration of residual sugars in the beverage by dilution.

24. The method of claim 23, wherein the dilution step is performed by adding water or a mixture of water and alcohol to the beverage.

25. The method of claim 23, wherein the concentration of residual sugars is decreased to about −1 to 7 Plato.

26. The method of claim 25, wherein the concentration of residual sugars is decreased to about 2 plato.
